(19) United States
(12) Patent Application Publication
(10) Pub. No.: US 2024/0344117 A1
(43) Pub. Date: Oct. 17, 2024

(54) A METHOD FOR SINGLE-CELL DNA SEQUENCING VIA IN SITU GENOMIC AMPLIFICATION AND COMBINATORIAL BARCODING

(71) Applicant: ARIZONA BOARD OF REGENTS ON BEHALF OF ARIZONA STATE UNIVERSITY, Scottsdale, AZ (US)

(72) Inventors: Kerry GEILER-SAMEROTTE, Scottsdale, AZ (US); Kara SCHMIDLIN, Mesa, AZ (US); Leandra BRETTNER, Mesa, AZ (US)

(21) Appl. No.: 18/683,200

(22) PCT Filed: Aug. 15, 2022
(86) PCT No.: PCT/US2022/040373

§ 371 Date: Feb. 12, 2024

Related U.S. Application Data
(60) Provisional application No. 63/233,177, filed on Aug. 13, 2021.

(51) Int. Cl. C12Q 1/6841; C12N 15/10; C12Q 1/686

(52) U.S. Cl. CPC C12Q 1/6841; C12N 15/1065; C12Q 1/686

(57) ABSTRACT

Disclosed herein is an in situ, high throughput, single-cell whole-genome sequencing technology developed for sequencing genomes in large heterogeneous cell populations. More specifically, the invention disclosed herein does not require cell sorting or isolation because it uses the cell membrane to separate each genome.

### CROSS-REFERENCE TO RELATED APPLICATIONS

This application claims priority to U.S. Appl. No. 63/233,177 filed Aug. 13, 2021, the entire content of which is incorporated herein by reference in its entirety.

### STATEMENT REGARDING FEDERALLY SPONSORED RESEARCH OR DEVELOPMENT

This invention was made with government support under R35GM133674 awarded by the National Institutes of Health by the National Science Foundation. The government has certain rights in the invention.

### FIELD

The field of the invention relates to methods for single-cell sequencing of genomic DNA.

### BACKGROUND

With the advent of Next Generation DNA sequencing and the “omics” era, technology for studying the genetic content and function of biological systems has rapidly advanced. Initially, genomics and transcriptomics studies were performed on populations or “batch cultures”. The resulting data represent an average across all cells. However, single-cell techniques permit the study of heterogeneity within populations, and are revealing the extent to which variation contributes to biological behaviors. In order to associate sampled genetic sequences with a given cell, the genetic material is often labelled with a DNA barcode sequence that is unique to each cell. In the first generation of single-cell technologies, barcodes were added after individual cells were sorted into separate containers, such as 100 uL wells or microfluidic droplets. Despite the difficulty of separating individual cells, no method for amplifying and barcoding genomic DNA from individual cells without physically separating cells currently exists. In particular, no method for amplifying and barcoding genomic DNA within individual cells currently exists.

### SUMMARY

Disclosed herein are methods for single-cell genomic DNA sequencing without physically separating cells, which is accomplished by using the cell itself as a container for the amplification and barcoding reactions. The method comprises a) dividing a plurality of fixed and permeabilized cells into a plurality of wells, each well comprising a first set of barcoding primers comprising: (i) a universal linker strand (ULS) sequence; wherein the primers in each well comprise the same ULS sequence; (ii) a first well-specific barcode (1-BC); wherein the primers in each well comprise a different 1-BC sequence; and a targeting region comprising at least one of: (iii) random hexamers sequences; wherein the random hexamers sequences hybridize to complementary sequences on genomic DNA of the cells; and (iv) specific sequences, wherein the specific sequences hybridize to target sequences on the genomic DNA of the cell; b) amplifying genomic DNA while it remains inside of each cell to create barcoded molecules under conditions that maintain cellular membrane integrity; c) pooling the cells from the plurality of wells; d) dividing the cells into a plurality of wells, each well comprising a second set of barcoding primers comprising: (v) an adapter sequence comprising a sequence complementary to the ULS sequence; (vi) a second well-specific barcode (2-BC); wherein the primers in each well comprise a different 2-BC sequence; and (vii) the ULS sequence wherein the primers in each well comprise the same ULS sequence; e) amplifying the barcoded molecules under conditions that maintain cellular membrane integrity; f) repeating steps d) through f) for a plurality of rounds, wherein the well-specific barcode is different in each round, and wherein in the final round, the set of barcoding primers further comprises an affinity moiety to generate barcoded amplicons comprising the affinity moiety; g) lysing the cells to release the barcoded amplicons comprising the affinity moiety; h) contacting the barcoded amplicons comprising the affinity moiety with a capture reagent; i) amplifying the barcoded amplicons off of the affinity moiety and affinity capture reagent to generate free amplification products; and optionally sequencing the free amplification products.

Also disclosed herein is a method comprising: a) dividing a plurality of fixed and permeabilized cells into a plurality of wells, each well comprising a first set of barcoding primers comprising: (i) a universal linker strand (ULS) sequence; wherein the primers in each well comprise the same ULS sequence; (ii) a first well-specific barcode (1-BC); wherein the primers in each well comprise a different 1-BC sequence; and a targeting region comprising at least one of: (iii) random hexamers sequences; wherein the random hexamers sequences hybridize to complementary sequences on genomic DNA of the cells; and (iv) specific sequences, wherein the specific sequences hybridize to target sequences on the genomic DNA of the cell; b) amplifying genomic DNA while it remains inside of each cell to create barcoded molecules under conditions that maintain cellular membrane integrity.

Also disclosed herein is a method comprising: a) capturing barcoded amplicons comprising an affinity moiety by contacting the amplicons with an affinity capture reagent; b) converting the barcoded amplicons into double-stranded captured amplicons; c) amplifying the double-stranded captured amplicons to generate free amplification products that are not attached to the affinity moiety and affinity capture reagent.

### BRIEF DESCRIPTION OF THE DRAWINGS

FIGS. 1A-D. Show an exemplary graphical representation of the method of the current disclosure. FIG. 1A illustrates the step of isothermally amplifying genomic DNA in situ. FIG. 1B illustrates the split and pool step. FIG. 1C illustrates the library preparation step. FIG. 1D illustrates the portions of sequence generated by the method of the current disclosure.

FIGS. 2A-F. Show representative experiments illustrating the outcome of the method of the current disclosure. FIGS. 2A and 2B are exemplary graphical representations of sequencing reads generated through the novel method of the disclosure aligned to the yeast genome. Multiple segments of the yeast genome are covered at varying read depths. FIG. 2C illustrates amplification of genomic yeast DNA in situ. FIGS. 2D-E illustrate amplification of a region of yeast DNA targeted by specific primers. FIG. 2F illustrates amplification of DNA from mammalian cells.

### DETAILED DESCRIPTION

Sequencing platforms are now capable of delivering enormous amounts of high-quality data. This allows for the possibility of sequencing the genomes of thousands of individual cells. However, current methods to isolate and tag single-cell genomes for sequencing are expensive and often require specialized equipment, or are arduous. The inventors developed a new method to sequence single-cell genomes that does not require cell isolation or specialized equipment beyond typical molecular biology laboratory standards, and thus is more user-friendly and scalable, allowing multiplexing of single cells from many different growth conditions or genetic backgrounds. Sequencing single cells has several advantages over sequencing pools of cells, including, but not limited to: identifying rare or low frequency mutations in a population, gaining a more detailed picture of microbes that inhabit specific environments, characterizing cells that all have unique DNA assortment, such as gametocytes, and determining the distribution of heterogeneous genomes in a population of cells, such as a tumor.

There is currently no existing technology or protocol that amplifies DNA inside of a cell in order to perform single-cell DNA sequencing. Most methods for single-cell sequencing physically isolate cells. The novel method combines in situ genome amplification with in situ combinatorial barcoding such that many cells from many conditions can all be processed in a single experiment without the need to physically isolate cells using specialized equipment. In other words, the novel method leverages the cell membrane to contain and separate the DNA from individual cells.

To the inventors' knowledge, no other disclosed work has used an isothermal polymerase, for example: phi29 polymerase, Klenow exo-DNA Polymerase I, Bsu polymerase, Bst polymerase, Bsm polymerase, to amplify the genome within the cell (3). In situ genome amplification is a critical first step to using combinatorial barcoding to uniquely label each cell. Combinatorial barcoding has been used quite successfully to perform single-cell RNA sequencing (1, 2), but has not yet been extended to single-cell DNA sequencing because the chemistry of DNA and RNA are different. While methods exist to convert genomic DNA to RNA inside of cells and prepare the resulting molecules for single-cell sequencing (12), these methods are arduous, have not been widely adopted, may have decreased fidelity relative to DNA-only preparations, and may be biased towards regions of the genome that are amenable to transcription. To the inventors' knowledge, no method exists to amplify DNA in situ and/or to capture barcoded DNA that was amplified in situ and prepare it for sequencing. RNA molecules do not need to be amplified in situ for single-cell experiments as those sequences are used to generate gene expression counts. In fact, amplification is a detriment, as it skews transcriptional profiles. DNA, however, must be amplified for genomic sequence analysis. The reason for this is that current sequencing technologies are error prone, thus having multiple unique copies of genomic regions allows us to distinguish true genetic differences or deviations from sequencing error.

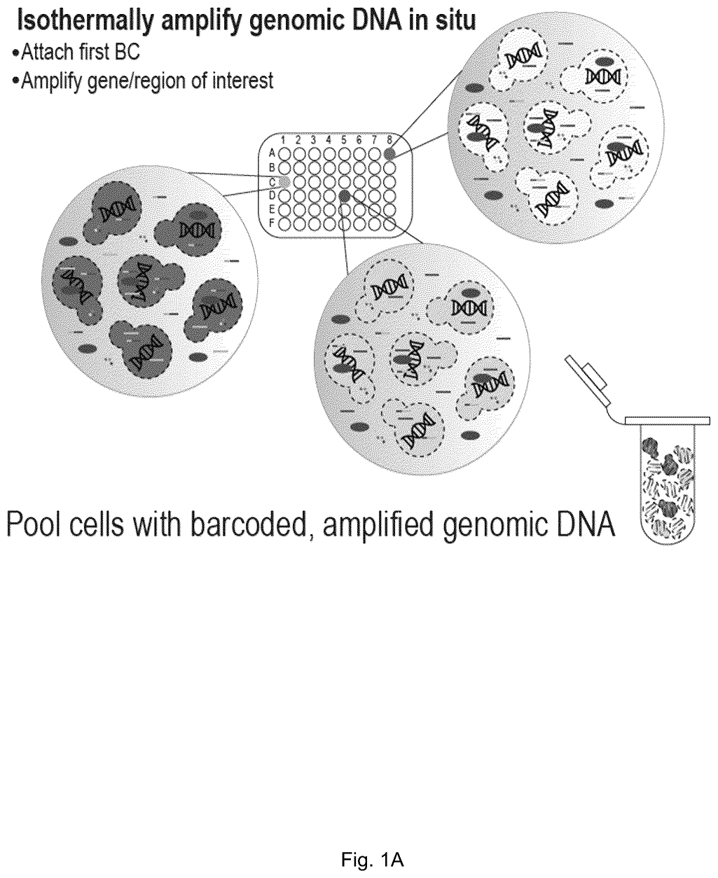

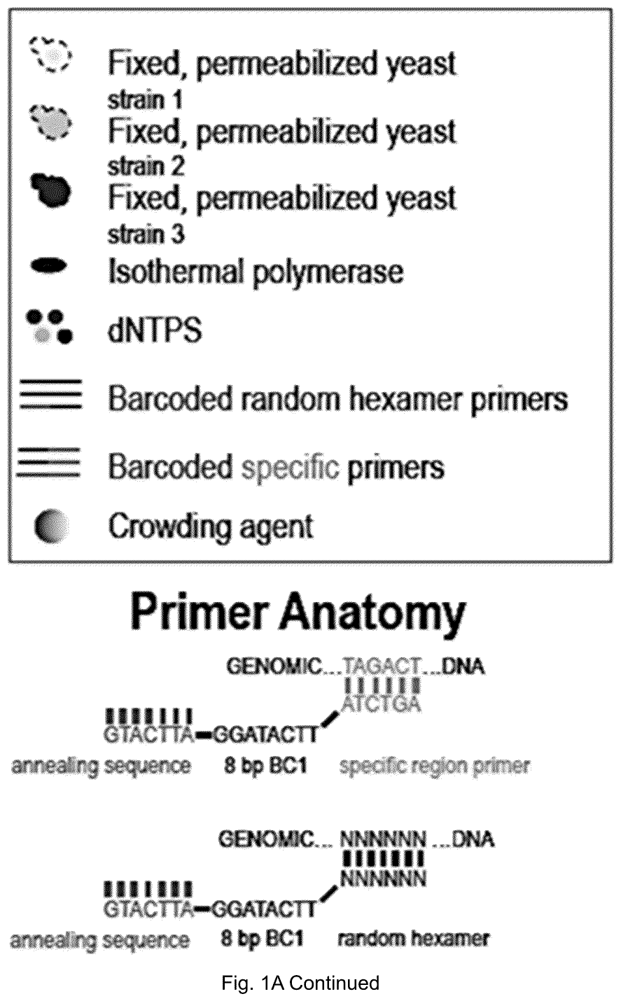

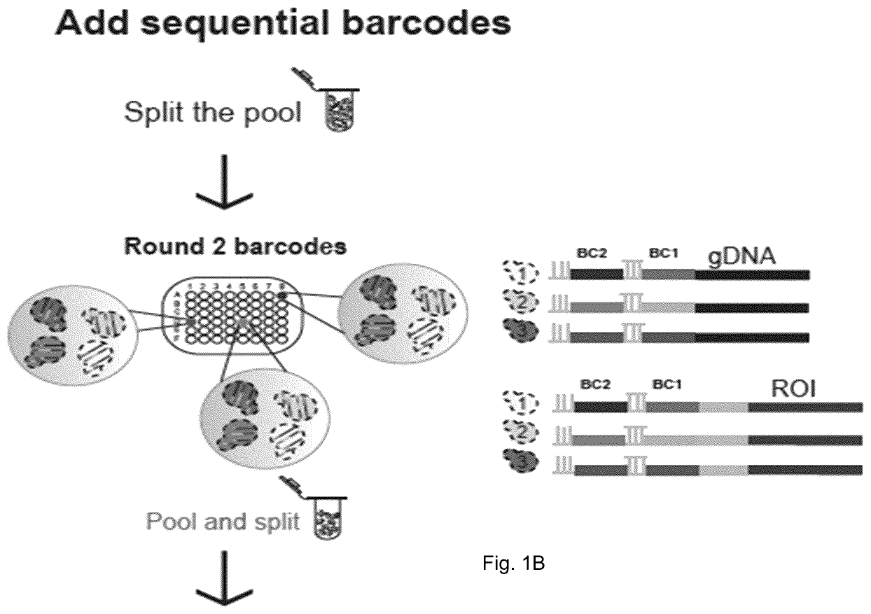

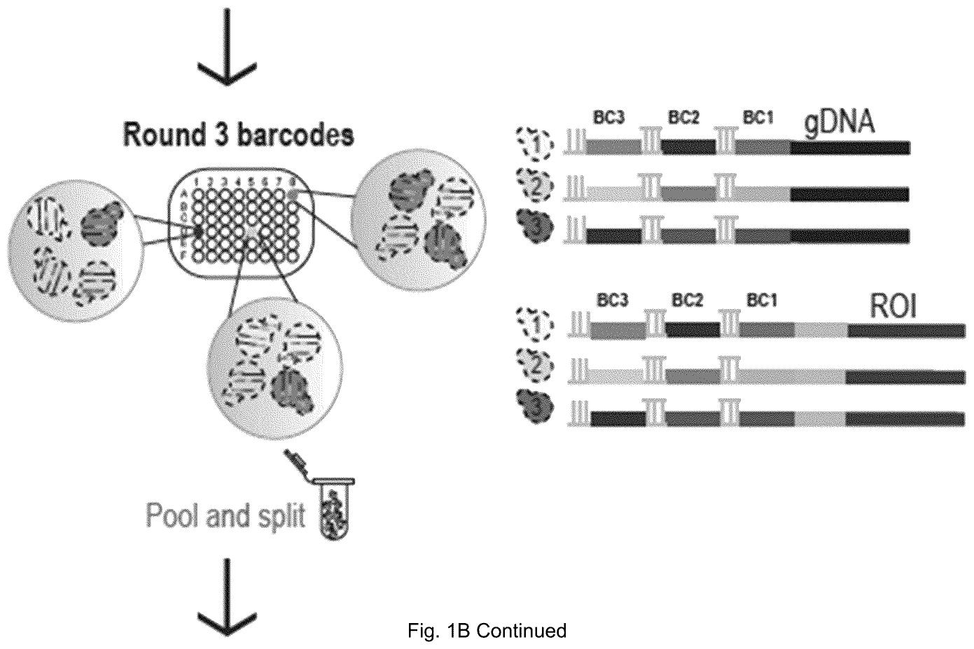

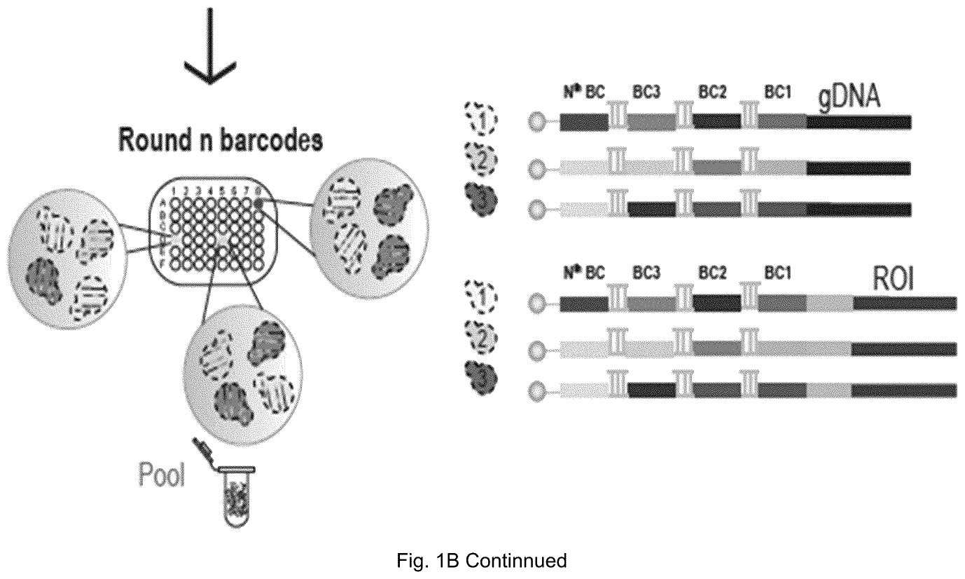

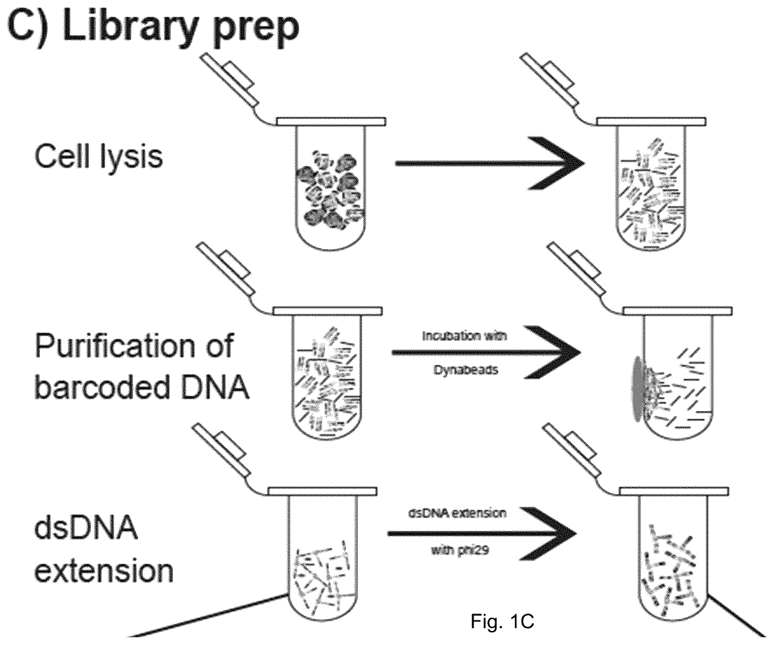

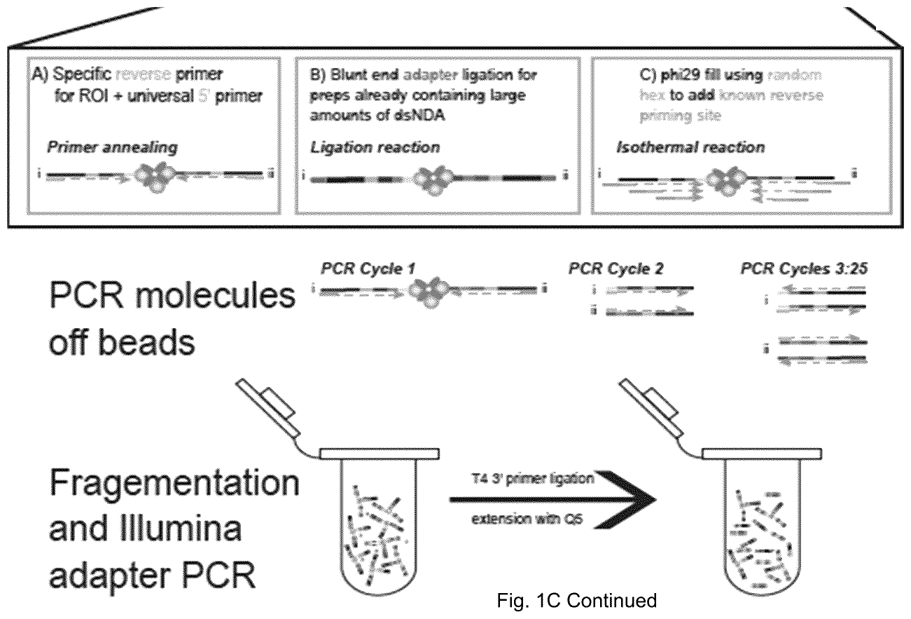

Originally developed for single-cell RNA sequencing, existing technology uses the cells as containers for their own transcriptional products (see references 1, 2, 4, 5, 6, 7, and 8, also see references U.S. Patent Pub. No. US20200263234A1, and U.S. Patent No. US10900065B2 and U.S. patent application Ser. No. 16/949,949). In such methods, cells are permeabilized so that barcodes can pass through holes in the cell membrane. These barcodes are added to the RNA sequences using an additive combinatorial strategy that randomly appends multiple barcodes from an existing pool, creating a unique combination that labels each cell's transcriptome in situ. Because of the different molecular chemistries and experimental needs for processing DNA, the inventors have needed to significantly modify combinatorial barcoding procedures used for RNA sequencing. The starting material is new (amplified DNA vs. RNA) and the post-processing methods are novel. For example, during post-processing the extracted barcoded DNA molecules must be appended with a 3′ primer adapter sequence. To append it, the DNA must all be made double-stranded to enable blunt end ligation. Thus, the inventors, for example, combine a secondary isothermal amplification reaction with random hexamer primers on the extracted bead-bound DNA with a ligation reaction to add the 3′ primer adapter. Three possible methods of amplifying bead-bound DNA are outlined in FIG. 1C.

Single-cell genomic sequencing technologies continue to improve; however, most protocols require individual cells to be mechanically separated by using either microfluidics or flow cytometry. In order to increase the number of cells that can be processed and the accessibility of these protocols, single cell genomic sequencing must move away from expensive equipment to reduce the cost per genome sequenced. In this disclosure, the inventors present an accessible and cost-effective method designed to sequence genomic DNA in heterogeneous cell populations without sorting cells. The method uses common lab equipment.

The impact of this technological advance will be to allow many fields greater access to single-cell genomic DNA sequencing. A few non-limiting potential impacts are listed below:

Tumors represent heterogeneous populations of cells. Sequencing the entire population can provide information about the common mutant lineages that exist in the tumor, but misses much of the diversity. Therefore, single cell methods are used to peer deeper into the tumor and see all the different types of mutations in it. However, the cost of sequencing scales linearly with every cell. The method allows sequencing the genomes of many single cells for a fraction of the cost. Further, it allows multiplexing of many samples at once, so the tumors of many patients can be analyzed at the same time without dramatically increasing cost. Since mammalian genomes are large, most single-cell sequencing methods can only analyze a small portion of each cell's genome. The technology allows targeting a portion of the genome of interest, for example, an oncogene, to dramatically increase the fraction of cells for which that region is sequenced.

The most precise method to study evolution in the laboratory involves barcoding the genome of model organisms such as yeast or bacterial cells and tracking the frequency of cell barcodes over time. (See, for example, (10)). This allows scientists to study millions of replicate evolution experiments, and see how many replicates develop adaptive mutations. However, it does not allow determination of the identity of the adaptive mutation. Previous work has identified adaptive mutations in evolved populations by manually picking out strains and determining if they have a barcode that is of interest. This is time consuming, expensive, and work intensive. The new single-cell genome sequencing technology avoids growing up and picking 1000's of individual colonies from evolved populations. Instead, the new technology allows one to grow the entire population from frozen stock, fix and permeabilize the cells and amplify each cell's genome in situ. This has the potential to reveal deep insights about the evolutionary process, specifically the identity of all possible mutations that are adaptive in a given environment. These data will have important practical applications, for example, towards predicting which mutations bacterial or yeast infections will develop in order to resist drug treatments. Since not 100% of the genome of every cell will be sequenced, the inventors target a portion of the genome containing the DNA barcode for higher coverage sequencing than the rest of the genome. This allows one to be sure to match genome sequences back to the barcode they are associated with so one can tell which mutations are adaptive.

There are many mixed microbial populations of interest, for example, the human microbiome or the inhabitants of soil or other ecological niches. Sequencing these populations in bulk creates challenges when sampling and assembling the genomes. Which genes belong to which genomes? Single-cell sequencing solves this by indicating which genes are found together in the same cell. This helps identify the full spectrum of species and strains that exist in these mixed microbial populations. However, current technology is limited not only in throughput, but also in the fact that some species are not amenable to isolation and/or their cell membranes cannot be broken down by a generic approach. The novel method does not require complete destruction of the cell membrane, in fact, it requires that it remain partially intact. Also, the method lends itself to multiplexing, so microbial samples can be divided, and different chemicals can be applied to each sample. This allows sequencing of genomes from a greater diversity of microbial species, at lower cost. The method gives a more complete picture of the composition of mixed microbial or bacterial populations.

Meiosis is the process by which egg and sperm cells are produced. It is error prone and different rates of errors, for example, missegregation of chromosomes, are associated with different chromosomes, different people, and different age groups. Current methods to study how these error rates vary across individuals and contribute to reproductive problems involve single-cell sequencing of sperm. This is accomplished by methods that isolate individual sperm cells via droplets. The method of the current disclosure increases throughput by not requiring the specialized machinery that isolates cells into droplets. The method of the current disclosure also would allow cells from multiple subjects to be analyzed simultaneously, without incurring additional cost.

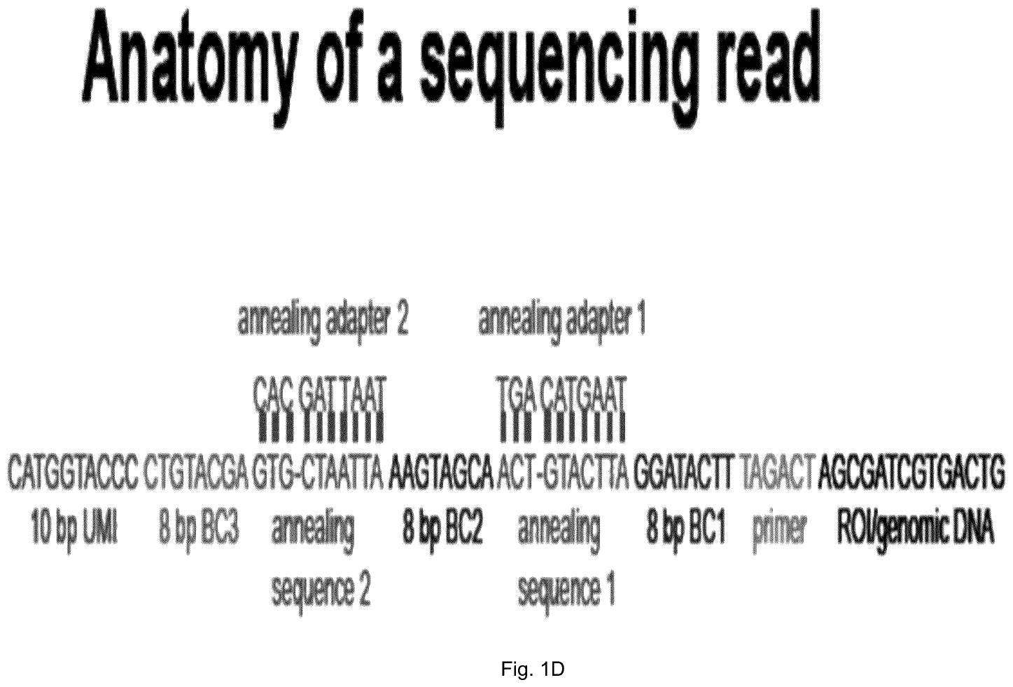

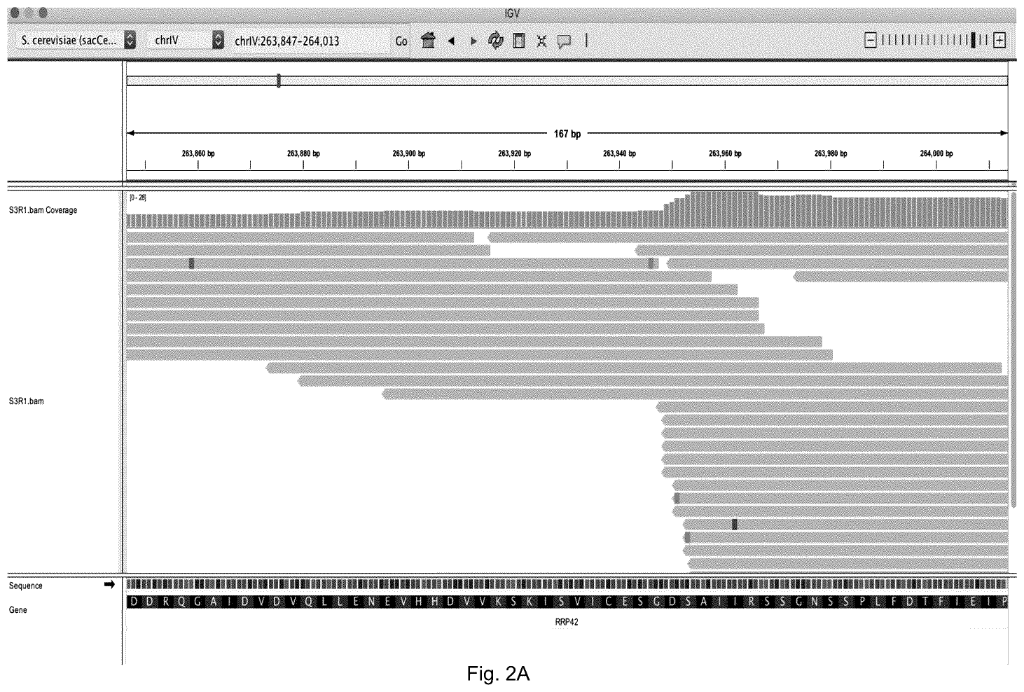

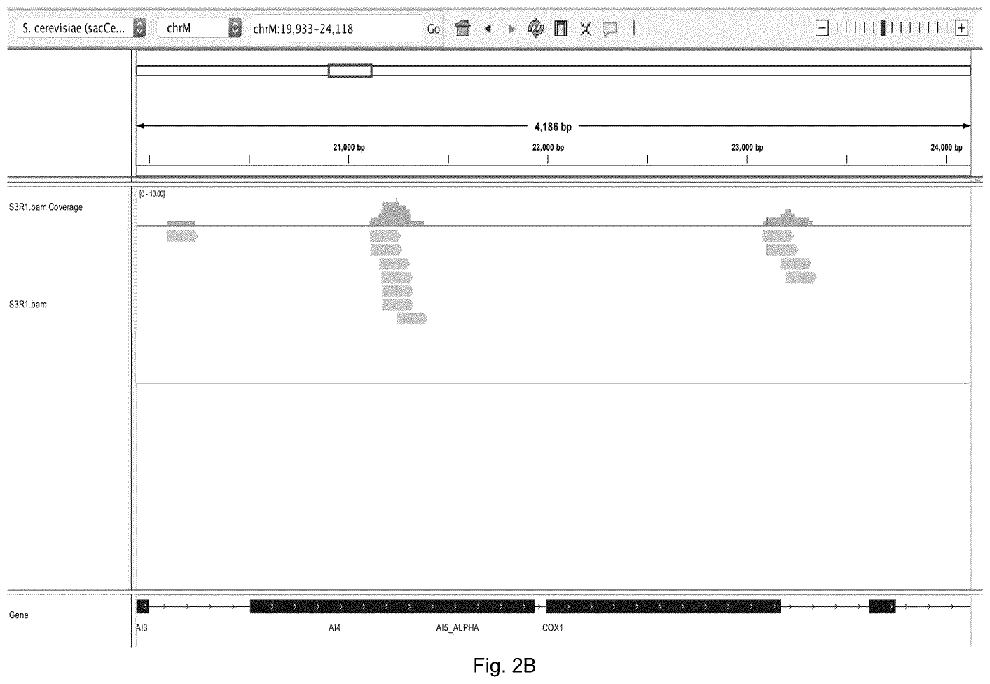

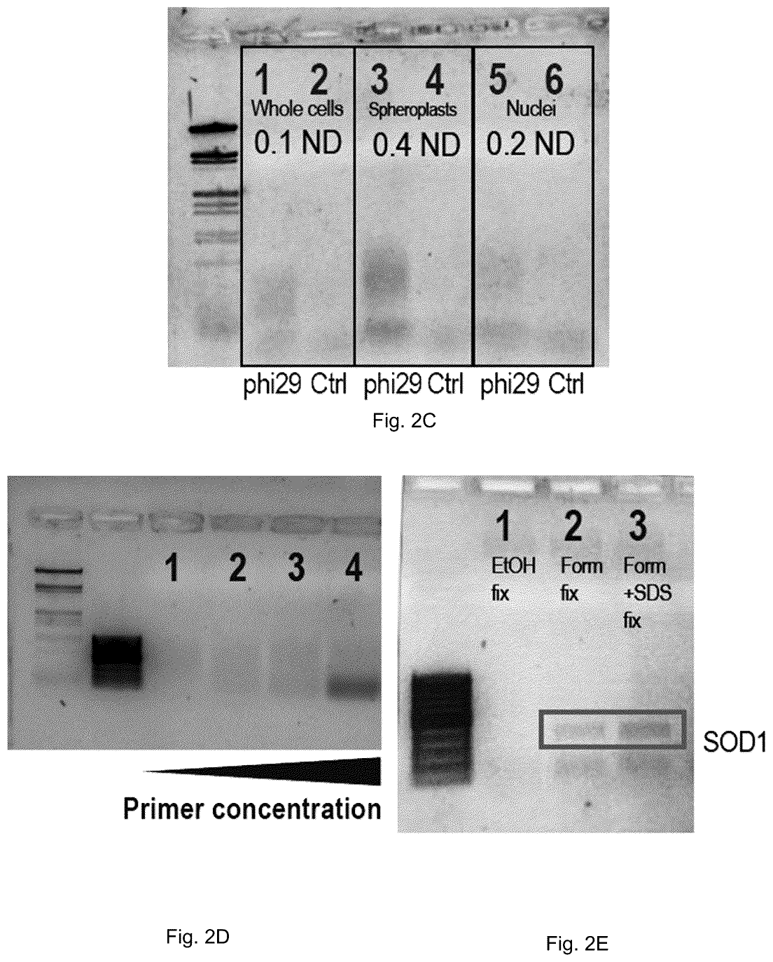

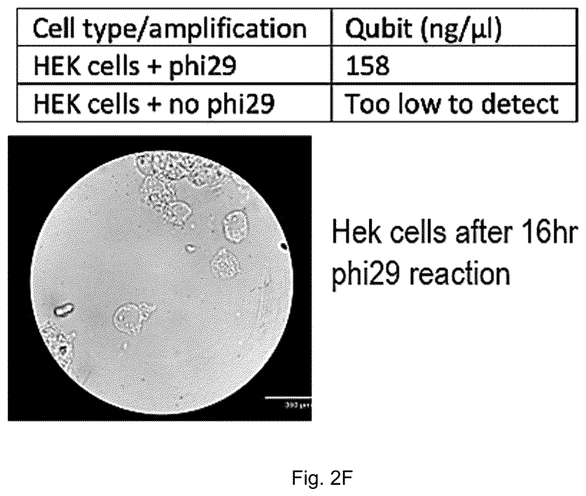

In opposition to microfluidics-based, single-cell sequencing methods that utilize isolated droplets or wells to contain the extracted DNA from a single cell, the invention of the current disclosure uses the cell itself as a container for its own DNA. Genomic DNA amplification and tagging are performed in situ. As sequencing requires many copies of DNA sequences, the first challenge is to amplify the genome. This is not an issue for RNA-seq because there are already many copies of each RNA inside the cell. Traditional polymerase chain reactions (PCR), typically used to amplify DNA, cannot be used in the invention of the current disclosure as the high temperatures required to denature the double helix would destroy the cell, and therefore the container for the reaction. To solve this problem, the inventors developed the novel method of the current disclosure. FIGS. 2C-2F provide data demonstrating that the novel method of this disclosure indeed solves this problem and allows amplification of genomic DNA in situ (FIGS. 2C and 2F) as well as amplification of a region of interest (FIGS. 2D and 2E) from various eukaryotic cells including yeast cells (FIGS. 2C-2E) and mammalian cells (FIG. 2F). Given DNA has identical chemistry across the tree of life, the novel method of this disclosure will also work on prokaryotic cells.

The following steps are illustrative in nature and not intended to limit the scope of the disclosure. To preserve the cellular components, the samples are formaldehyde fixed overnight. Cells are then permeabilized so that membranes can allow enzymes and other reagents to pass into the cell to access the genomic DNA. After the cells are fixed and permeabilized, DNA is denatured in situ through temperature or chemical means to open chromatin to allow for better primer and polymerase binding. Genomic DNA is then amplified in situ via an isothermal polymerase. In some embodiments the isothermal polymerase is one or more of phi29 polymerase, Klenow exo-DNA Polymerase I, Bsu polymerase, Bst polymerase, Bsm polymerase. In some embodiments, the isothermal polymerase can effectively strand displace and copy DNA at low temperatures (e.g., a temperature lower than required for strand denaturation). In some embodiments, these reactions are performed in a multi-well plate with each well containing random hexamer primers that bind many places in the genome. The random hexamers contain a well-specific barcode and a ubiquitous annealing sequence at the 5′ end for further barcoding post-amplification. The first barcode sequence serves as a conditional signifier, because all cells that originate from that initial well are intentionally loaded there. Thus, dozens of separate samples, e.g., cells from different experimental conditions or different subjects, can be processed together (FIG. 1A). In some embodiments, the reactions are performed in a multi-well plate with each well containing specific primers that bind to specific target genes of interest (GOI) in the genome. The specific primers contain a well-specific barcode and a ubiquitous annealing sequence at the 5′ end for further barcoding post-amplification. The first barcode sequence serves as a conditional signifier, because all cells that originate from that initial well are intentionally loaded there. Thus, dozens of separate samples, e.g., cells from different experimental conditions or different subjects, can be processed together (FIG. 1A).

The next challenge is adding additional barcodes such that every single cell ends up with a unique combination (FIG. 1B). After the genome is tagged and amplified, the cells are pooled and split into another multi-well plate where each well contains a short, unique barcode sequence with a complementary adapter to the ubiquitous annealing sequence. A T4 ligation reaction covalently bonds these barcodes to the 5′ end of each cell's amplified DNA. The cells are subsequently pooled and split into a new plate where the process is repeated, adding a second barcode to the first. Thus, cells that received the same first barcode are unlikely to receive the same second barcode. This process is completed an arbitrary number of times depending on the size of the population of cells being processed, as unique barcode combination possibilities scale exponentially with each additional round (e.g. 96 barcodes in a 96 well plate, n split-pools=96ⁿ possible barcode combinations). Each cell is, thus, uniquely labelled by probabilistically biasing the outcome such that it takes its own path through the barcode plates. The terminal barcode addition from the last round of split pooling is tagged with a biotin molecule so that the successfully barcoded sequences can be selectively segregated.

At this point in the method, each cell contains amplified and barcoded copies of its genomic DNA. The cells are then lysed to extract the genetic material and incubate with streptavidin coated magnetic beads to extricate properly barcoded sequences (FIG. 1C). All other material is washed away. The resulting DNA molecules present challenges, namely that they are now affixed to the substrate comprising the capture reagent, e.g., a bead. Currently available sequencing library preparation methods are unable to solve these problems. While the beads serve to isolate the desired molecules, in order to sequence this DNA, the molecules must be copied off of the beads, as they are firmly attached via the biotin-streptavidin bond. For RNA-seq this is done by starting with a template switch reaction, but this method is incompatible for use with amplified genomic DNA because the chemistry is different. Copying the DNA off of these beads represents a major challenge that the inventors solved. FIGS. 2A and 2B provide data demonstrating that the novel method of this disclosure can indeed capture and sequence bead bound DNA from the yeast genome. In the novel method, the DNA is copied off the beads following one of three procedures depending on the circumstance (FIG. 1C). In embodiments where the DNA represents known regions of the genome, this is done by annealing primers that target that region, similarly to the terminal primer sequence (FIG. 1C; leftmost box labeled “primer annealing”) to generate double-stranded captured DNA. Thus, in such embodiments, addition of the terminal primer sequence may not be necessary. In some embodiments, generating double-stranded captured amplicons comprises ligating a double-stranded DNA sequence comprising the terminal primer sequence to the free end of the captured amplicons. In such embodiments, a non-barcoded reverse primer or a barcoded reverse primer is used in the in situ gDNA amplification step (FIG. 1A) to generate more template, thereby resulting in captured amplicons that are mostly double-stranded. In these embodiments, or in embodiments where the DNA represents the entire genome, DNA amplicons are copied off the substrate, e.g., beads, by attaching an intermediate 3′ primer adapter via blunt-end ligation to the unbarcoded end (FIG. 1C; middle box labeled “ligation reaction”). In some embodiments, the DNA is copied off the beads by using random hexamer primers with an attached primer adapter region and performing a phi29 (or other isothermal polymerase) reaction on the beads (FIG. 1C; rightmost box labeled “isothermal reaction”). In some embodiments, the random hexamer primers include a terminal primer sequence. Since phi29 and several other isothermal polymerases are strand displacing, the longest complementary strand will be annealed to the bead-attached DNA. Washing the beads removes excess or short copies. In some embodiments, to amplify all molecules of DNA off the beads, traditional exponential PCR is performed with primers that target 1) the adapter region in the cell-specific barcode and 2) the regions added or described in FIG. 1C, e.g. the terminal primer sequence. Then, in some embodiments, the libraries are prepared using standard practices because these copies, unlike the originals, are not bound to a streptavidin bead (FIG. 1C). The portions of a sequencing read generated by the methods of the present disclosure is illustrated in FIG. 1D.

Many methods including, but not limited to, lineage tracking, metagenomic studies, tumor resistance profiling and VDJ regions from immune cells would benefit from incorporating single-cell genomic data. Another powerful tool in the innovative single-cell sequencing method is the ability to amplify, barcode, and trace back precise regions in the genome to an individual cell within a heterogeneous population. This is useful because single-cell technologies do not sequence the entire genome of every cell. They are only able to sequence pieces of every genome, and then utilize clustering approaches. The method of the current disclosure allows for enrichment of genomic regions of interest to ensure that they are amplified in a larger fraction of cells. Amplification with isothermal polymerase traditionally utilizes random hexamer primers, however, site-specific first step barcoded primers can be designed to tag any genomic area of interest, for example, a particular oncogene. Here the inventors disclose the use of a combination of random and site-specific primers to gain power to detect specific unique genomic details.

The present invention is described herein using several definitions, as set forth below and throughout the application.

### Definitions

The disclosed subject matter may be further described using definitions and terminology as follows. The definitions and terminology used herein are for the purpose of describing particular embodiments only and are not intended to be limiting.

The term “subject” may be used interchangeably with the terms “individual” and “patient” and includes human and non-human subjects. In some embodiments, subjects may be plants, fish, birds, reptiles, or mammals. In some embodiments, the disclosed methods are performed on fungal, bacterial, archaeal, or protozoal cells.

As used herein, “fixation” or “fixing” refers to the process of chemically stabilizing organic, inorganic, or a combination of organic and inorganic molecules through the use of reagents, known as “fixatives”. Exemplary fixatives for the present disclosure include, but are not limited to, formaldehyde, formaldehyde derived from paraformaldehyde, formalin, phosphate buffered formalin, formal calcium, formal saline, zinc formalin, alcoholic formalin, glutaraldehyde, other organic aldehydes, methanol, ethanol, isopropanol, or other organic alcohols, or solutions containing organic alcohols or aldehydes.

As used herein, “permeabilization” or “permeabilizing” refer to the process of introducing openings into barriers to allow the penetration of desired molecules past the aforementioned barrier. In some embodiments, the barrier comprises a cell membrane, and or a cell wall. In some embodiments, permeabilization is performed by, for example, enzymes on biological membranes. Exemplary enzymes for permeabilization of biological membranes include, but are not limited to: proteinase K and zymolyase. In some embodiments, permeabilization is performed by, for example, detergents on biological membranes.

The term “hybridization,” as used herein, refers to the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which hybridization of fully complementary nucleic acid strands is strongly preferred are referred to as “stringent hybridization conditions” or “sequence-specific hybridization conditions”. Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair composition of the oligonucleotides, ionic strength, and incidence of mismatched base pairs, following the guidance provided by the art (see, e.g., Sambrook et al., 1989, Molecular Cloning—A Laboratory Manual, Cold Spring Harbor Laboratory, Cold Spring Harbor, New York; Wetmur, 1991, Critical Review in Biochem. and Mol. Biol. 26 (3/4): 227-259; and Owczarzy et al., 2008, Biochemistry, 47:5336-5353, which are incorporated herein by reference).

As used herein, “amplification” refers to the process of semi-conservatively replicating nucleic acid strands by enzyme-catalyzed extension. Exemplary enzymes for amplification of nucleic acids in the current disclosure include, for example, nucleic acid polymerases. In some embodiments, an isothermal polymerase is used to amplify nucleic acids. In some embodiments, amplification is carried out with a high-fidelity polymerase, such as Q5, with the technique known as the polymerase chain reaction (PCR). Amplification can be performed with natural and non-natural nucleotide bases, ribonucleotide bases or deoxyribonucleotide bases, labeled nucleotide bases, and the like.

As used herein, “isothermal amplification” describes amplification of DNA targets without heat denaturation of DNA. In contrast, polymerase chain reaction (PCR) requires cycling through different temperatures for denaturation, hybridization, and extension. Isothermal amplification may be preceded by a higher temperature hybridization step that does not denature the DNA target. Exemplary polymerases useful for isothermal amplification are referred to herein as isothermal polymerases, and include, but are not limited to phi29 polymerase, Klenow exo-DNA Polymerase I, Bsu polymerase, Bst polymerase, Bsm polymerase.

As used herein, “ligation” refers to the joining of two nucleic acid molecules through the formation of covalent phosphodiester bonds. Ligation may involve the joining of double-stranded or single stranded nucleic acid molecules. In some embodiments, two blunt-ended nucleic acid duplexes are ligated together. In some embodiments, two nucleic acid duplexes that have single-stranded regions that are substantially complementary to one another allowing hybridization of the two nucleic acid duplexes are ligated to one another.

As used herein, “pooling” refers to the process of taking previously separate samples, such as cells, and combining them to create a “pool” of samples (such as cells) that optionally may be separated bioinformatically and identity determined post-experiment during data analysis.

As used herein, the noun “well” refers to a single container or reaction vessel. Though the term well is often used when referring to plates or microplates, it is to be understood that the methods of the current disclosure may also be performed using, for example, tubes or other vessels capable of containing and separating liquids.

As used herein, “affinity moiety” refers to a chemical constituent, often attached to a molecule of interest that can be specifically recognized and bound by a “capture reagent” with high affinity, and with binding strength suitable to allow purification of the molecule of interest to which the affinity moiety is attached. The use of affinity moieties with capture reagents is collectively referred to as “affinity capture”, in the context of separation of molecules of interest using the pair of reagents (affinity capture reagents). In some embodiments, exemplary affinity capture reagents include, without limitation, for example, biotin and streptavidin, digoxigenin and anti-digoxigenin antibodies, antibody-antigen pairs, and covalent click chemistry.

As used herein, “sequencing” refers to the sequencing of nucleic acids. Sequencing of nucleic acids may be accomplished using, by way of example but not by way of limitation, Sanger sequencing, or next-generation sequencing.

As used herein, “barcode” refers to a nucleotide sequence of any length that is used to identify, for example, nucleotide sequences that are derived from a single sample. An exemplary property of a barcode is the ability to distinguish the sequence of the barcode from any known sequence present in the sample, thereby rendering the barcode sequence informatically distinct and permitting identification or quantification of any nucleotide sequence comprising the barcode. In some embodiments, a barcode may be 6-8 nucleotides in length. Each barcode must be detected in a single sequencing “read.” Therefore, barcode length is, in principle, dictated by the sequencing platform used to analyze the samples.

As used herein, “universal linker strand” or “ULS” refers to a nucleotide sequence that facilitates the hybridization of single stranded primers, such that the hybridization partner of the ULS is the reverse complement of the ULS, or substantially similar to the reverse complement of the ULS. In some embodiments, the ULS is 10-20 (inclusive) nucleotides in length. In some embodiments, the ULS is 15 nucleotides in length. The “universal linker strand” may also be referred to as the “ubiquitous annealing sequence.”

As used herein, “terminal primer sequence” refers to a sequence that is known and can be used to anneal a primer for amplification. Thus, addition of a terminal primer sequence to an amplicon allows amplification of the amplicon by addition of a primer complementary to the terminal primer sequence.

As used herein, “split and pool” refers to a process for introducing complexity into a group of compounds such that the knowledge of the initial source of each compound is preserved and can be determined after the completion of the split and pool process (see references 1, 2, 4, 5, 6, 7, 8, also see references U.S. Patent Pub. No. US20200263234A1, and U.S. Patent No. US10900065B2 and U.S. patent application Ser. No. 16/949,949 Split and pool relies on probability to ensure that each individual compound has a high statistical likelihood to take a unique path through a set of steps, with each step introducing a new “barcode” which is linked to the compound. A first “barcoding event”, meaning the attachment, such as by ligation, of a barcode to the compound, is performed with a knowledge of the identity of the compound and the identity of the barcode to which each compound is attached. After each barcoding event, all of the individual compounds are combined, or “pooled”, and “split”, or redistributed into new reaction vessels, with each vessel containing a unique barcode. Thus, a second round of barcoding reduces the chances that two compounds will be split into the same reaction vessel and be attached (e.g., ligated) to the same barcode. Therefore, after successive rounds of splitting and pooling the compounds, each of the compounds is likely to be attached (e.g., ligated) to a unique set of barcodes that correspond to the compounds unique trajectory through the split and pool process. The possible number of unique compounds that can be effectively barcoded using split and pool increases with both the number of reaction vessels, and therefore the number of barcodes, and with the number of successive rounds of barcoding events. Non-limiting examples of potential uses for the split and pool process include the preparation of nucleic acid libraries. As disclosed herein, in an embodiment of the present technology, split and pool may be used to efficiently label nucleic acids that are derived from a single cell with a unique barcode allowing for multiplexed sequencing of nucleic acids derived from many cells.

As used herein, the term “random hexamer” or “random hexonucleotide” refers to a region of six nucleotides in length comprising sequences that are synthesized at random. The purpose of random hexamers is, in most applications, to bind complementarily to nucleotide sequences of unknown identity. Thus, because random hexamers theoretically cover all possible sequence permutations for a hexameric (6-member) nucleotide, they are likely to bind at many positions to nucleotides of any sequence. It should be understood, however, that a key feature of random hexamers is not that they are six nucleotides in length, but rather that they have random sequence identity. In other words, for many applications it is possible to provide random pentamers (5-member), heptamers (7-member), or other random sequences in place of hexamers. In some embodiments, a random hexamer comprises a part of, or a portion of a larger oligonucleotide, such as an oligonucleotide primer.

As used herein, “primer” refers to a single-stranded nucleotide. In some embodiments, a primer is used to initiate semi-conservative replication of nucleic acids. In some embodiments, primers are used to “barcode” nucleic acid sequences of interest. By way of example, an oligonucleotide primer may comprise from 5′ to 3′: a universal linker strand, a barcode and a random hexamer sequence. By way of example, an oligonucleotide primer may comprise from 5′ to 3′: a universal linker strand, a random hexamer sequence, and a barcode. In an embodiment of the novel method of the disclosure, the primers that are used to randomly barcode genomic DNA are random hexamer primers. The barcodes used are 8 bp long and the UCLs are 15 bps (e.g., UCL1-BC1-random hexamer, ATCCACGTGCTTGAG-ACTCGTAA-NNNNNNATAAGC). The specific primer used to amplify a specific region of interest is currently 21 bp long (e.g., UCL1-BC1-TTAATATGGACTAAAGGAGGC). Primers of other lengths may be acceptable.

As used herein, “crowding agent” refers to compounds that decrease the solvent available to macromolecules, thereby increasing the relative concentration of said macromolecules and altering their properties. In some applications, crowding agents have the effect of increasing enzyme activity and accelerating reactions resulting in faster and potentially more specific assays. In some embodiments, crowding agents may include one or more of polyethylene glycol (PEG), polyethylene glycol 8000 (PEG-8000), trehalose, and sorbitol. In some embodiments crowding agents may include ficoll or dextrans.

The terms “nucleic acid” and “nucleic acid molecule,” as used herein, refer to a compound comprising a nucleobase and an acidic moiety, e.g., a nucleoside, a nucleotide, or a polymer of nucleotides. Nucleic acids generally refer to polymers comprising nucleotides or nucleotide analogs joined together through backbone linkages such as but not limited to phosphodiester bonds. Nucleic acids include deoxyribonucleic acids (DNA) and ribonucleic acids (RNA) such as messenger RNA (mRNA), transfer RNA (tRNA), etc. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or include non-naturally occurring nucleotides or nucleosides. Furthermore, the terms “nucleic acid,” “DNA,” “RNA,” and/or similar terms include nucleic acid analogs, i.e. analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadeno sine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

Nucleic acids, proteins, and/or other compositions described herein may be purified. As used herein, “purified” means separate from the majority of other compounds or entities, and encompasses partially purified or substantially purified. Purity may be denoted by a weight by weight measure and may be determined using a variety of analytical techniques such as but not limited to mass spectrometry, HPLC, spectrophotometer, etc.

As used herein, the terms “complementary” or “complementarity” are used in reference to “polynucleotides” and “oligonucleotides” (which are interchangeable terms that refer to a sequence of nucleotides) related by the base-pairing rules. For example, the sequence “5′-C-A-G-T,” is complementary to the sequence “5′-A-C-T-G.” Complementarity can be “partial” or “total.” “Partial” complementarity is where one or more nucleic acid bases is not matched according to the base pairing rules. “Total” or “complete” complementarity between nucleic acids is where each and every nucleic acid base is matched with another base under the base pairing rules.

As used herein, the term “specific to” is used to define the relationship between macromolecular binding partners. For example, as used above, two nucleotide sequences that possess total complementarity to one another would be considered “specific” for one another, i.e., each totally complementary nucleotide would be specific to the other.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polyribonucleotides can also be prepared enzymatically. Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In the context of the present disclosure, the following abbreviations for the commonly occurring nucleic acid bases are used. “A” refers to adenine, “C” refers to cytosine, “G” refers to guanine, “T” refers to thymine, and “U” refers to uracil. The aforementioned abbreviations may also be used to refer to nucleosides or nucleotides comprising the nucleic acid bases. For example, “G” may refer guanine, guanosine, or guanidine, depending on the context.

As used in this specification and the claims, the singular forms “a,” “an,” and “the” include plural forms unless the context clearly dictates otherwise. For example, the term “a substituent” should be interpreted to mean “one or more substituents,” unless the context clearly dictates otherwise.

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover, the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or ‘B or “A and B.”

All language such as “up to,” “at least,” “greater than,” “less than,” and the like, include the number recited and refer to ranges which can subsequently be broken down into ranges and subranges. A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

### Exemplary Embodiments

Provided below are several, non-limiting exemplary embodiments of the methods disclosed herein.

1. A method comprising a) dividing a plurality of fixed and permeabilized into a plurality of wells, each well comprising a first set of barcoding primers comprising: (i) a universal linker strand (ULS) sequence; wherein the primers in each well comprise the same ULS sequence; (ii) a first well-specific barcode (1-BC); wherein the primers in each well comprise a different 1-BC sequence; and a targeting region comprising at least one of: (iii) random hexamers sequences; wherein the random hexamers sequences hybridize to complementary sequences on genomic DNA of the cells; and (iv) specific sequences, wherein the specific sequences hybridize to target sequences on the genomic DNA of the cell; b) amplifying genomic DNA while it remains inside of each cell to create barcoded molecules under conditions that maintain cellular membrane integrity; c) pooling the cells from the plurality of wells; d) dividing the cells into a plurality of wells, each well comprising a second set of barcoding primers comprising: (v) an adapter sequence comprising a sequence complementary to the ULS sequence; (vi) a second well-specific barcode (2-BC); wherein the primers in each well comprise a different 2-BC sequence; and (vii) the ULS sequence wherein the primers in each well comprise the same ULS sequence; e) amplifying the barcoded molecules under conditions that maintain cellular membrane integrity; f) repeating steps d) through f) for a plurality of rounds, wherein the well-specific barcode is different in each round, and wherein in the final round, the set of barcoding primers further comprises an affinity moiety to generate barcoded amplicons comprising the affinity moiety; g) lysing the cells to release the barcoded amplicons comprising the affinity moiety; h) contacting the barcoded amplicons comprising the affinity moiety with a capture reagent; and i) amplifying the barcoded amplicons off of the affinity moiety and affinity capture reagent to generate free amplification products.

2. The method of embodiment 1, wherein the target region comprises specific sequences, and wherein the specific sequences include forward primers and reverse primers for the target sequence; and wherein the method further comprises ligating a double-stranded DNA sequence comprising a terminal primer sequence to a free end of the barcoded amplicons before amplifying the barcoded amplicons off of the affinity moiety and affinity capture reagent.

3. The method of embodiment 1, wherein the barcoding primers comprise specific sequences, and wherein the method further comprises converting the barcoded amplicons into double-stranded amplicons by contacting the barcoded amplicons with a polymerase, and amplification primers that hybridize to segments of the barcoded amplicons complementary to the specific sequences; and performing an amplification reaction.

4. The method of embodiment 1, further comprising converting the barcoded amplicons into double-stranded amplicons by contacting the barcoded amplicons with a polymerase, and oligonucleotides, wherein the oligonucleotides comprise random hexamers and a terminal primer sequence, wherein the oligonucleotides are configured to produce double-stranded barcoded amplicons comprising the terminal primer sequence.

5. The method of embodiment 1, wherein the barcoding primers comprise only random hexamer sequences.

6. The method of embodiment 1, wherein the barcoding primers comprise only specific sequences.

7. The method of embodiment 1, wherein the barcoding primers comprise both random hexamer sequences and specific sequences.

8. The method of embodiment 1, wherein the amplification of step b) comprises an isothermal amplification reaction.

9. The method of embodiment 8, wherein the temperature of the isothermal amplification reaction is about 20-40° C.

10. The method of embodiment 8, wherein the temperature of the isothermal amplification reaction is about 20-30° C.

11. The method of embodiment 8, wherein the temperature of the isothermal amplification reaction is about 30-40° C.

12. The method of claim 8, wherein the temperature of the isothermal amplification reaction is about 30° C.

13. The method of any one of embodiments 1-12, wherein in step b) the cells are incubated for about 12-24 hours.

14. The method of any one of embodiments 1-12, wherein in step b) the cells are incubated for about 16 hours.

15. The method of embodiment 8, wherein step b) comprises contacting the plurality of fixed and permeabilized cells with an isothermal polymerase.

16. The method of embodiment 15, wherein the isothermal polymerase is phi29.

17. The method of embodiment 16, wherein the concentration of isothermal polymerase is about 400 units/ml.

18. The method of any one of embodiments 1-12, wherein step b) comprises contacting the plurality of fixed and permeabilized cells with a crowding agent.

19. The method of embodiment 18, wherein the crowding agent comprises one or more of: polyethylene glycol 8000 (PEG-8000), trehalose, and sorbitol.

20. The method of embodiment 19, wherein the crowding agent is PEG-8000.

21. The method of embodiment 19, wherein the concentration of PEG-8000 is about 7.5% volume/volume.

22. The method of embodiment 19, wherein the crowding agent is trehalose.

23. The method of embodiment 22, wherein the concentration of trehalose is about 0.4 M.

24. The method of embodiment 19, wherein the crowding agent is sorbitol.

25. The method of embodiment 24, wherein the concentration of sorbitol is about 0.5 M.

26. The method of any one of embodiments 1-12, wherein the adapter sequence is ligated to the ULS sequence with T4 DNA ligase.

27. The method of any one of embodiments 1-12, wherein lysing the plurality of cells comprises contacting the cells with sodium dodecylsulfate (SDS).

28. The method of any one of embodiments 1-12, wherein lysing the plurality of cells comprises contacting the cells with proteinase K.

29. The method of any one of embodiments 1-12, wherein the affinity moiety comprises biotin and the capture reagent comprises streptavidin.

30. The method of any one of embodiments 1-12, wherein the affinity moiety comprises digoxigenin and the capture reagent comprises anti-digoxigenin antibody.

31. The method of embodiment 3 or 4, wherein the step of converting the barcoded amplicons into double stranded amplicons comprises performing an isothermal amplification reaction.

32. The method of embodiment 31, wherein the polymerase is an isothermal polymerase.

33. The method of embodiment 32, wherein the concentration of the isothermal polymerase is about 400 units/ml.

34. The method of embodiment 31, wherein the temperature of the isothermal amplification reaction is about 20-40° C.

35. The method of embodiment 31, wherein the temperature of the isothermal amplification reaction is about 20-30° C.

36. The method of embodiment 31, wherein the temperature of the isothermal amplification reaction is about 30-40° C.

37. The method of embodiment 31, wherein the temperature of the isothermal amplification reaction is about 30° C.

38. The method of embodiment 31, wherein the isothermal amplification reaction is performed for about 30-120 minutes.

39. The method of embodiment 3 or 4, wherein the step of converting the barcoded amplicons into double-stranded amplicons further comprises contacting the barcoded amplicons with a crowding agent.

40. The method of embodiment 39, wherein the crowding agent is selected from the group consisting of: polyethylene glycol 8000 (PEG-8000), trehalose, and sorbitol.

41. The method of embodiment 40, wherein the crowding agent is PEG-8000.

42. The method of embodiment 41, wherein the concentration of PEG-8000 is about 7.5% volume/volume.

43. The method of embodiment 40, wherein the crowding agent is trehalose.

44. The method of embodiment 43, wherein the concentration of trehalose is about 0.4 M.

45. The method of embodiment 40, wherein the crowding agent is sorbitol.

46. The method of embodiment 45, wherein the concentration of sorbitol is about 0.5 M.

47. The method of any one of embodiments 1-12, wherein amplifying the barcoded amplicons in step i) comprises performing polymerase chain reaction (PCR).

48. The method of embodiment 47, wherein primers that hybridize to the terminal primer sequence are used in the PCR.

49. The method of embodiment 47, wherein primers that hybridize to the target sequences are used in the PCR.

50. The method of any one of embodiments 1-12, further comprising 1) purifying the free amplification products.

51. The method of embodiment 50, wherein the free amplification products) are purified using solid phase reversible immobilization (SPRI) selection.

52. The method of any one of embodiments 1-12, further comprising sequencing the free amplification products.

53. The method of any one of embodiments 1-12, wherein the ULS sequence is different in each round, and wherein the adapter in each round is complementary to the ULS sequence of the previous round.

54. A method comprising: a) dividing a plurality of fixed and permeabilized cells into a plurality of wells, each well comprising a first set of barcoding primers comprising: (i) a universal linker strand (ULS) sequence; wherein the primers in each well comprise the same ULS sequence; (ii) a first well-specific barcode (1-BC); wherein the primers in each well comprise a different 1-BC sequence; and a targeting region comprising at least one of: (iii) random hexamers sequences; wherein the random hexamers sequences hybridize to complementary sequences on genomic DNA of the cells; and (iv) specific sequences, wherein the specific sequences hybridize to target sequences on the genomic DNA of the cell; b) amplifying genomic DNA while it remains inside of each cell to create barcoded molecules under conditions that maintain cellular membrane integrity.

55. The method of embodiment 54, wherein the targeting region comprises only random hexamer sequences.

56. The method of embodiment 54, wherein the targeting region comprises only specific sequences.

57. The method of embodiment 54, wherein the targeting region comprises both random hexamer sequences and specific sequences.

58. The method of any one of embodiments 54-57, wherein the amplification of step b) comprises an isothermal amplification reaction.

59. The method of embodiment 58, wherein the temperature of the isothermal amplification reaction is about 20-40° C.

60. The method of embodiment 58, wherein the temperature of the isothermal amplification reaction is about 20-30° C.

61. The method of embodiment 58, wherein the temperature of the isothermal amplification reaction is about 30-40° C.

62. The method of embodiment 58, wherein the temperature of the isothermal amplification reaction is about 30° C.

63. The method of embodiment 58, wherein in step b) the cells are incubated for about 12-24 hours.

64. The method of embodiment 58, wherein in step b) the cells are incubated for about 16 hours.

65. The method of embodiment 58, wherein step b) comprises contacting the plurality of fixed and permeabilized cells with an isothermal polymerase.

66. The method of embodiment 65, wherein the concentration of isothermal polymerase is about 400 units/ml.

67. The method of any one of embodiments 54-57, wherein step b) comprises contacting the plurality of fixed and permeabilized cells with a crowding agent.

68. The method of embodiment 67, wherein the crowding agent is selected from the group consisting of: polyethylene glycol 8000 (PEG-8000), trehalose, and sorbitol.

69. The method of embodiment 68, wherein the crowding agent is PEG-8000.

70. The method of embodiment 69, wherein the concentration of PEG-8000 is about 7.5% volume/volume.

71. The method of embodiment 68, wherein the crowding agent is trehalose.

72. The method of embodiment 71, wherein the concentration of trehalose is about 0.4 M.

73. The method of embodiment 68, wherein the crowding agent is sorbitol.

74. The method of embodiment 73, wherein the concentration of sorbitol is about 0.5 M.

75. A method comprising: a) capturing barcoded amplicons comprising an affinity moiety by contacting the amplicons with an affinity capture reagent; b) converting the barcoded amplicons into double-stranded captured amplicons; c) amplifying the double-stranded captured amplicons to generate free amplification products that are not attached to the affinity moiety and affinity capture reagent.

76. The method of embodiment 75, wherein converting the barcoded amplicons into double-stranded captured amplicons comprises contacting the barcoded amplicons with primers that hybridize to a specific sequence on the amplicons, and a DNA polymerase.

77. The method of embodiment 75, wherein the primers comprise a terminal primer sequence, and wherein after step c) the double-stranded captured amplicons comprise the terminal primer sequence.

78. The method of embodiment 75, wherein converting the barcoded amplicons into double-stranded captured amplicons comprises contacting the captured bar-coded amplicons with a polymerase, and oligonucleotides; wherein the oligonucleotides comprise random hexamers, and a terminal primer sequence; and wherein the oligonucleotides are configured to produce double-stranded captured amplicons comprising the terminal primer sequence.

79. The method of embodiment 75, wherein converting the barcoded amplicons into double-stranded captured amplicons comprises ligating a double-stranded DNA sequence comprising a terminal primer sequence to a free end of the barcoded amplicons.

80. The method of any one of embodiments 75-79, wherein the double-stranded captured amplicons are amplified using polymerase chain reaction (PCR), and wherein the PCR reaction uses an amplification primer that is complementary to the terminal primer sequence.

81. The method of any one of embodiments 75-79, wherein the affinity moiety comprises biotin and the affinity capture reagent comprises streptavidin.

82. The method of any one of embodiments 75-79, wherein the affinity moiety comprises digoxigenin and the affinity capture reagent comprises anti-digoxigenin antibody.

83. The method of embodiment 75, wherein converting the barcoded amplicons into double-stranded captured amplicons comprises contacting the barcoded amplicons with an isothermal polymerase.

84. The method of embodiment 83, wherein the concentration of isothermal polymerase is about 400 units/ml.

85. The method of embodiment 88, wherein the amplification of step b) comprises an isothermal amplification reaction.

86. The method of embodiment 85, wherein the temperature of the isothermal amplification reaction is about 20-40° C.

87. The method of embodiment 85, wherein the temperature of the isothermal amplification reaction is about 20-30° C.

88. The method of embodiment 85, wherein the temperature of the isothermal amplification reaction is about 30-40° C.

89. The method of embodiment 85, wherein the temperature of the isothermal amplification reaction is about 30° C.

90. The method of any one of embodiments 75-79, wherein step b) is performed for about 30-120 minutes.

91. The method of any one of embodiments 75-79, wherein step b) comprises contacting the barcoded amplicons with a crowding agent.

92. The method of embodiment 91, wherein the crowding agent is selected from the group consisting of: polyethylene glycol 8000 (PEG-8000), trehalose, and sorbitol.

93. The method of embodiment 92, wherein the crowding agent is PEG-8000.

94. The method of embodiment 93, wherein the concentration of PEG-8000 is about 7.5% volume/volume.

95. The method of embodiment 92, wherein the crowding agent is trehalose.

96. The method of embodiment 95, wherein the concentration of trehalose is about 0.4 M.

97. The method of embodiment 92, wherein the crowding agent is sorbitol.

98. The method of embodiment 97, wherein the concentration of sorbitol is about 0.5 M.

99. The method of any one of embodiments 75-79, wherein step c) comprises amplification of the double-stranded captured amplicons using polymerase chain reaction (PCR).

100. The method of any one of embodiments 75-79, further comprising purifying the free amplification products.

101. The method of embodiment 100, wherein the free amplification products are purified using solid phase reversible immobilization (SPRI) selection.

102. The method of any one of embodiments 75-79, further comprising sequencing the free amplification products.

103. A method comprising amplifying genomic DNA while it remains inside of a fixed and permeabilized cell to create amplification products under conditions that maintain cellular membrane integrity.

104. A method comprising amplifying a specific region of genomic DNA while it remains inside of a fixed and permeabilized cell to create amplification products under conditions that maintain cellular membrane integrity.

105. A kit for amplifying genomic DNA within cells, the kit comprising: a) a first plurality of barcoding primers comprising: (i) a universal linker strand (ULS) sequence; wherein each of the plurality of primers comprises the same ULS sequence; (ii) a first barcode (1-BC); wherein each of the plurality of primers comprises a different 1-BC sequence; and a targeting region comprising at least one of: (iii) random hexamers sequences; wherein the random hexamers sequences hybridize to complementary sequences on genomic DNA; and (iv) specific sequences, wherein the specific sequences hybridize to target sequences on genomic DNA; b) a second plurality of barcoding primers comprising: (v) an adapter sequence comprising a sequence complementary to the ULS sequence; (vi) a second barcode (2-BC); wherein each of the plurality of primers comprises a different 2-BC sequence; and wherein each set comprises a different plurality of barcoding primers; and (vii) the ULS sequence each of the plurality of primers comprises the same ULS sequence; c) a third plurality of barcoding primers comprising: (viii) an adapter sequence comprising a sequence complementary to the ULS sequence; (ix) a third barcode (3-BC); wherein each of the plurality of primers comprises a different 3-BC sequence; (x) the ULS sequence each of the plurality of primers comprises the same ULS sequence; and (xi) an affinity moiety.

### EXAMPLES

The following Examples are illustrative and should not be interpreted to limit the scope of the claimed subject matter.

### Example 1—SPiNgen Protocol

Some sections of this non-limiting example protocol are adapted from publicly available protocols for yeast spheroplast generation (11) and Split-Pool Ligation based Transcriptomics sequencing in mammalian and bacterial cells (7, 8). The method of the current disclosure may be performed using several techniques for generating fixed and permeabilized cells and for ligating adapters to DNA sequences known in the art. However, for illustrative purposes the following example protocol is presented.

### Prepare the DNA Barcoding Plates for Later Use

By way of example but not by way of limitation, methods for preparing plates loaded with adapters for ligation are well known in the art. See, e.g., references 7 and 8, incorporated by reference herein in their entirety. Briefly, barcoding plates were generated using the following method. Plates were loaded with barcode and linking oligos. For each ligation plate the barcode and linker oligonucleotides were annealed with the following thermocycling protocol:
1. Heat to 95 C for 2 minutes
2. Ramp down to 20 C for at a rate of −0.1 C/s
3. 4 C

### 1. Fixation and Permeabilization of Yeast Cells

Methods of creating spheroplasts from yeast cells are well known in the art. The following method, adapted from Kiseleva et al. (11), incorporated herein by reference in its entirety, was used to generate spheroplasts. However, one of skill in the art will recognize that other techniques will be suitable to generate spheroplasts. In addition, though this example generates fixed and permeabilized yeast cells, one of skill in the art will recognize that fixing and permeabilizing cells any type of cell (e.g., vertebrate, mammalian, insect, reptile, bird, bacterial, etc.) are known in the art, using e.g., formalin/formaldehyde and detergent compositions. Thus, this general method step of generating fixed and permeabilized cells may be substituted for methods suited to the particular cell type of interest without adversely affecting the novel method disclosed herein.

The following solutions were prepared:
Buffer 1 (30 mL/3 g of cells):
   M Tris-HCl (3 mL 1M sol)
   M DTT in dH2O pH 9.4 (46.2759 mg DTT, 27 mL dH2O)
Buffer 2 (˜100 mL/3 g of cells):
   M Sorbitol (21.8604 g)
   M potassium phosphate (424.532 mg)
   mM magnesium chloride (4.7606 mg)
   dH2O (100 mL)
   Buffer 3/4 (1 mL per sample)
   uL zymolyase in 1 mL buffer 2

Briefly, by way of example but not by way of limitation, spheroplasts were created by growing yeast cells to an appropriate density, pelleted, incubated in buffer 1 in a 50-ml plastic centrifuge tube for 25 min at 30° C. with moderate shaking, pelleted, resuspended in buffer 2, pelleted, and resuspended in buffer 3, pelleted and resuspended in buffer 3 or 4, and checked by microscope for the formation of spheroplasts. A spheroplast is a cell lacking or deficient in the cell wall and the whole having a spherical form. Once 70% of the cells had become spheroplasts, the cells were pelleted and resuspended in buffer 2, pelleted again and repeat this step two more times to remove the enzyme.

### Fixation

Briefly, by way of example but not by way of limitation, the following method was used to fix the spheroplasts of the previous step. The following solutions were made (note that, where possible, molecular grade reagents were used):
10 mL of a 4% formalin (1080 uL of 37% formaldehyde solution+8.92 mL PBS) solution and store at 4 C (or on ice).
2200 uL of 1×PBS (on ice)
2200 uL of 100 mM Tris pH 8.0 (on ice)
1758.24 uL of Y sol 1 (0.1M NazEDTA, 1M sorbitol, PH ˜7.5)
1760 uL of 0.005 U/uL zymolyase: Y sol 1 (1.76 uL 5 U/uL zymolyase to 1758.24 uL Y sol 1)
2200 uL 0.04% Tween
50 mL PBS (on ice)

Cells were pelleted and resuspended in cold formalin. Samples were stored in a 4 C fridge for ˜18 hours.

### Permeabilization

Cells were centrifuged at 3000 G for 5 minutes and resuspended in 100 mM Tris. Cells were pelleted again, and supernatant removed. The zymolyase: Y sol 1 permeabilization reagent was mixed with cells and incubated at 37 deg for 15 min. Cells were then pelleted and re-suspended in 0.04% Tween. Cells were permeabilized on ice for 3 minutes. One of skill in the art will understand that, in this exemplary experiment, the non-ionic detergent Tween is used to permeabilized cells. However, other detergents are known in the art and may be substituted for Tween, for example Triton. Cells were pelleted and resuspended in 500 uL cold 1×PBS.

### 1. Fixation and Permeabilization of Mammalian Cells

Methods of fixation and permeabilization of mammalian cells are well known in the art. The following method was used to fix and permeabilize human embryonic kidney cells for the purpose of demonstrating successful DNA amplification in situ in mammalian cells (results in FIG. 2F). The following solutions were made (note that, where possible, molecular grade reagents were used):
10 mL 1.33& formalin (360 uL of 37% formaldehyde solution+9.66 mL 1×PBS) stored on ice or at 4 C.
6 mL of 1×PBS.
2 mL of 0.5×PBS.
500 uL of 5% Triton-X100
500 uL of 100 mM Tris HCl pH 8.0

Cells were centrifuged at 500 G for 3 minutes at 4 C. The supernatant was aspirated and cells were resuspended in 1 mL of cold 1×PBS and the cells were kept on ice. The resuspended cells were passed through a 40 uM filter to help disrupt aggregates. 3 mL of cold 1.33% formalin was added and cells were fixed on ice for 10 minutes. 160 uL of 5% Triton-X100 was added to the cell fixative mixture, and the cells were permeabilized on ice for 3 minutes. The cells were pelleted and resuspended in 500 uL of 100 mM Tris-HCl, 500 uL 1×PBS and 20 uL 5% Triton-X100. The cells were pelleted again and resuspended in 300 uL of cold 0.5×PBS.

### 2. DNA Denaturation and Primer Hybridization (Day 2)

Several methods exist for DNA denaturation including heat incubation, but a method using formamide treatment was performed and is described here. The following solutions were prepared:

### Primer Annealing Mix (Per Reaction):

5 uL 10 uM primer (random hexamer or specific region)
20 uL 20×SSC buffer
30 uL 100% formamide
55 uL molecular grade water

Cells were pelleted and resuspended in 100 μL of the primer annealing mix. They were then incubated at 37 C for 2.5 hours. 1 mL of cold 1×PBS was added to the 100 uL primer annealing mix to wash, and the cells were pelleted. The 1.1 mL of wash solution was aspirated and the cells were again resuspended in 1 mL of cold 1×PBS to wash again. The cells were pelleted and resuspended in 300-500 uL cold 1×PBS depending on cell density.

3. Amplify Genomic DNA and/or Gene of Interest Inside of Cells (Day 2)

The following novel method of step 2 is presented as an example protocol that the inventors have successfully reduced to practice to amplify genomic DNA in situ using the model system of brewer's yeast. 8 uL of the first barcode primer stock was added into the top 4 rows (48 wells) of a new 96 well plate. The plate was covered with an adhesive plate seal until ready for use.

The following isothermal polymerase mix was prepared on ice at volumes sufficient to generate a total of 12 uL per reaction: 2.5 uL of 10× isothermal polymerase buffer, 0.2 uL of 20 mg/mL BSA, 2.5 uL 10 mM (per base) dNTPs, 1 uL 400 U/mL isothermal polymerase, 5.8 uL crowding agent (27% PEG8000, 1.8M trehalose, or 2M sorbitol).

12 uL of the isothermal polymerase mix was added to each of the top 48 wells. Each well thus contained a volume of 20 uL. Cells were vortexed and 5 uL of cells in 1×PBS were added to each of the top 48 wells, for a volume of 25 uL per well. Plates were placed into a thermocycler with the following protocol: (a) 30 C for 16 hrs; (b) 4 C forever. Each isothermal polymerase reaction was transferred to a 5 mL tube (on ice), and 9.6 uL of 10% Triton-X100 was added to get a final concentration of 0.1%. Pooled isothermal polymerase reactions were centrifuged for 5 min at 3000 G. Supernatant was aspirated and the cells were resuspended into 2 mL of PBS. Cells were then vortexed on high for 1 minute, filtered through a 40 um pluriStrainer (checked on microscope), and then vortexed on high for 1 minute immediately prior to the addition of the ligation mix.

4. Ligation of Barcodes that Track Single Cells

The following method of barcoding cells is adapted from references 7 and 8. However, it will be apparent that other methods to barcode cells are well known in the art. Briefly, by way of example but not by way of limitation, the following ligation master mix of was created: 770 uL molecular grade water, 500 uL 10×T4 ligase buffer, 750 uL 50% PEG8000, 20 uL 400 U/uL T4 DNA ligase. Cells were added to the master mix and incubated. The round 2 blocking solution was made as follows: 316.8 uL 100 uM BC_0340 (7, 8), 300 uL 10×T4 ligase buffer, 538.2 uL molecular grade water.

The cells were then split and pooled and ligated to the round 2 barcodes. The round 2 blocking solution is added to the wells, and incubated. The cells were then split, pooled, and ligated to the round 3 barcodes, wherein the barcodes now comprised the affinity moiety biotin. Finally, the round 3 blocking solution was added to the cells comprised of: 369 uL 100 uM BC_0066 (7, 8), 800 uL 0.5M EDTA, and 2031 uL molecular grade water.

Next, ligation was terminated and the cells were washed in wash buffer (4000 uL 1×PBS and 40 uL 10% Triton X-100) and cells were allocated to sublibraries. The cells were then lysed and the DNA was prepared for sequencing. Many methods of lysing cells are known in the art and it will be apparent to one of skill in the art that substitutions to the reagents used in the method of references 7 and 8 may be made.

By way of example, 2× lysis buffer was made as follows (50 uL per sublibrary): 1 uL 1M Tris-HCl pH 8, 4 uL 5M NaCl, 10 uL 0.5M EDTA, 22 uL 10% SDS, 13 uL molecular grade water.

The cells in each sublibrary were lysed in the lysis buffer with 5 uL 20 mg/ml proteinase K for 2 hours at 55 C.

### Purification of Barcoded DNA:

The following example protocol for purification of barcoded DNA was used. However, it will be apparent to one of skill in the art that substitutions may be made to the following protocol that will not negatively affect the ability of the artisan to practice the method. Furthermore, additional methods of DNA purification of barcoded DNA are well known in the art and the following method is presented as a non-limiting example. Briefly, 5 uL 100 mM PMSF was added to the lysate to terminate the proteinase K. Then, MyOne C1 Dynabeads were incubated with the lysate and allowed to bind to the streptavidin. The lysates were then washed with buffers containing Tris-HCL pH 8, NaCl, EDTA, and nuclease free water to remove unbound DNA.

### 5. Extension of Bead-Bound Barcoded DNA to Generate Double-Stranded Product:

The following novel method is presented as an example protocol that the inventors have successfully reduced to practice to extend bead bound DNA using the model system of brewer's yeast. The following isothermal polymerase mix was prepared per sample: 5 uL 10× isothermal polymerase buffer, 0.5 uL 20 mg/mL BSA, 5 uL 10 mM (per base) dNTPs, 2 uL isothermal polymerase, 2 uL 10 uM random hexamer primers, 35.5 uL 2M sorbitol.

Samples were placed against a magnetic rack and until liquid cleared. With sample still on magnetic rack, supernatant was removed and the samples were washed with 250 uL of water. Samples were the resuspended in 50 uL of isothermal polymerase mix and incubated for 1 hour at 30 C.

### Adaptor Ligation to Allow Amplification of DNA Off Beads (Day 3 or 4):

### Anneal Adapters

The following mix was added to a thermocycler at −0.1 C/s from 95 C to 20 C:
a. 1 uL 1M NaCl
b. 9.5 uL BC_108a (100 uM) (7, 8)
c. 9.5 uL BC_109 (100 uM) (reverse complement of BC_108a)

### Adapter Ligation

The adapter ligation mix was made as follows per reaction: 17.5 uL nuclease free water, 20 uL WGS Enzymatics ligation buffer, 10 uL WGS Enzymatics DNA ligase, 2.5 uL annealed adapters.

Samples were placed against a magnetic rack until the liquid becomes clear. Supernatant was removed and the beads were washed with 250 uL of water, and resuspended in 50 uL of water. 50 uL of the adapter mix was added to the 50 uL sample for a 100 uL reaction volume. Samples were incubated at 20° C. for 15 minutes (lid temperature 30° C.). To stop the reaction, samples can again be placed against a magnetic rack until liquid becomes clear. Samples can then be resuspended in 250 uL Tris-Tween and stored at 4 C for no more than 2 days.

### Amplification Bead-Bound DNA to Create Bead-Free Copies:

The following PCR mix was prepared per sample: 121 uL Kapa HiFi 2× master mix, 9.68 uL 10 uM BC_0108 and BC_0062 (7, 8), 101.64 uL nuclease free water.

Samples were placed against a magnetic rack until liquid became clear. Samples were washed with 250 uL nuclease-free water. Samples were resuspend with 220 uL PCR mix and split equally into 4 different PCR tubes. The following thermocycling program was then run: (a) 95 C 3 min; (b) 98 C 20 s; (c) 65 C 45 s; (d) 2 C 3 min; Repeat (b-d) 19×(20 total cycles); 4 C hold.

The resulting products can be run on an agarose gel or otherwise analyzed. There will likely be a combination of DNA and dimer present.

### SPRI Size Selection (0.8×):

PCR reactions were combined into a single tube. 180 uL of the pooled PCR reaction was removed and placed in new 1.7 mL tube. 144 uL of Kapa Pure Beads were added to tube and vortexed briefly to mix. Samples were incubated for 5 min to bind DNA. Tubes were then placed against a magnetic rack and until liquid becomes clear. Supernatant was removed, and beads were washed 2× with 750 uL 85% ethanol. Ethanol was removed and the beads were air dried bead (˜5 min). Dry beads were then resuspended in 20 uL of water. Once beads were fully resuspended in the water, samples were incubate the tube at 37 C for 10 min. Tubes were then placed against a magnetic rack and until liquid cleared. 18.5 uL of eluant was transferred into a new optical grade PCR tube, and a bioanalyzer trace was run on 10 uL of the eluant. If no dimer is present after size selection, move directly to the next section. If dimer is still present, go back to the previous section and perform another size selection.

### 6. Standard NGS Library Preparation:

Following the novel amplification of bead-bound DNA, the free barcoded genomic DNA sequences were used to create libraries for sequencing according to methods known in the art. At this point, the sublibraries exist as pools of DNA fragments that can be prepared following the user's preferred NGS library preparation protocols. Sequencing adapters may be added using the WGS fragmentation and ligation protocol as described in Kuchina and Brettner et al. (8), tagmentation, etc. One of skill in the art will appreciate that many methods for preparation of libraries for sequencing and sequencing thereof may be substituted for the method described in (8).

### Results

An experiment was conducted using the novel method of this disclosure to amplify genomic yeast DNA while it remained inside of intact yeast cells using random hexamer primers, appending single-cell barcodes, capturing that barcoded DNA, copying the DNA off the capture beads, and sequencing the barcoded DNA. To sequence the barcoded DNA fragments that resulted from this experiment, an Illumina paired-end sequencing run with at least 86 bp in Read 2 was used.

### Results of Yeast Genome Sequencing Experiment:

The inventors have successfully barcoded 163 Saccharomyces cerevisiae cells and retrieved genomic coverage of one or more regions of all 16 chromosomes and mitochondrial DNA. FIGS. 2A and 2B show IGV images showing sequencing reads obtained using the novel method do indeed align to the yeast genome. Multiple segments are covered at varying read depths. Thus, the novel method of this disclosure can amplify DNA in situ, append single-cell barcodes to that DNA, capture that DNA on beads, copy that DNA off beads, and prepare it for sequencing.

In situ DNA amplification experiments: Since to the inventor's knowledge, DNA has not been previously amplified in situ, the inventors performed several experiments to confirm that this was possible in a variety of cell types and for a variety of genomic regions.

### Results of Amplification Studies:

FIG. 2C shows gel images and corresponding Qubit (DNA concentration) values for whole yeast cells, yeast spheroplasts, and yeast nuclei treated via the novel method of this disclosure, or treated via a control protocol that lacks isothermal polymerase. Lanes 1, 3, and 5 of this agarose gel show dark coloration corresponding to amplified DNA, while control wells 2, 4, and 6 (which did not contain any isothermal polymerase) do not show any DNA amplification. The numbers in each lane provide measurements of DNA concentration taken via Qubit, which are non-zero for lanes 1, 3, and 5, while no DNA is detected in lanes 2, 4, and 6 (ND stands for “none detected”. Since more DNA was visible in the experiments including polymerase than in the controls (FIG. 2C), the inventors conclude that the method is successful at amplifying DNA. FIGS. 2D and 2E suggest that the novel method is also successful at amplifying genetic regions of interest from yeast cells. The agarose gels show bands corresponding to the anticipated size of the region of interest targeted by specific primers, suggesting that the region of interest was amplified in situ. Bands of the expected size are present after in situ reactions were performed using primers that target a specific gene. FIG. 2F reports Qubit DNA concentration data for washed mammalian cells treated with or without phi29 to demonstrate successful in situ amplification. The table reports DNA concentration after performing the novel method on HEK cells. DNA was detected after lysing permeabilized cells that had been treated with isothermal polymerase, but no DNA was detected in a control experiment lacking the polymerase. Microscopy images in FIG. 2F show that the mammalian cells remained intact after the amplification protocol, suggesting the amplification proceeded in situ.

Thus, all the data in FIG. 2 combined indicates that the inventors have demonstrated that the method of the current disclosure has been successfully reduced to practice.

### REFERENCES

1. A. B. Rosenberg*, C. M. Roco*, R. A. Muscat, A. Kuchina, P. Sample, Z. Yao, L. Gray, D. J. Peeler, S. Mukherjee, W. Chen, S. H. Pun, D. L. Sellers, B. Tasic, G. Seelig, Single-cell profiling of the developing mouse brain and spinal cord with split-pool barcoding. Science, eaam8999 (2018). https://science.sciencemag.org/content/360/6385/176 abstract
2. A. Kuchina*, L. M. Brettner*, L. Paleologu, C. M. Roco, A. B. Rosenberg, A. Carignano, R. Kibler, M. Hirano, R. W. DePaolo, G. Seelig, Microbial single-cell RNA sequencing by split-pool barcoding. Science. 371 (2021), doi: 10.1126/science.aba5257. https://science.sciencemag.org/content/371/6531/eaba5257/tab-article-info
3. A. C. Payne*, Z. D. Chiang*, P. L. Reginato*, S. M. Mangiameli, E. M. Murray, C. Yao, S. Markoulaki, A. S. Earl, A. S. Labade, R. Jaenisch, G. M. Church, E. S. Boyden, J. D. Buenrostro, F. Chen, In situ genome sequencing resolves DNA sequence and structure in intact biological samples. Science. 371 (2021). https://science-sciencemag.org.ezproxy1.lib.asu.edu/content/sci/371/6532/eaay3446 full.pdf
5. US20200263234A1 Entitled: “IN SITU COMBINATORIAL LABELLING OF CELLULAR MOLECULES”
6. US10900065B2 Entitled: “METHODS AND KITS FOR LABELLING CELLULAR MOLECULES”
7. patent application Ser. No. 16/949,949 filed Nov. 20, 2020 Entitled: “A METHOD FOR PREPARATION AND HIGH-THROUGHPUT MICROBIAL SINGLE CELL RNA SEQUENCING OF BACTERIA” Inventors: Georg Seelig, Anna Kuchina, Leandra Brettner, & William DePaolo
8. Rosenberg and Roco et al. Science. 13 Apr. 2018: Vol. 360, Issue 6385, pp. 176-182.
9. Kuchina and Brettner et al. Science. 2021 Feb. 19; 371 (6531).
10. Levy, Blundell, Venkataraman, Petrov, Fisher, Sherlock. Quantitative evolutionary dynamics using high resolution lineage tracing. Nature. 2015 Mar. 12; 519 (7542): 181-6.
11. Kiseleva, Allen, Rutherford, Murray, Morozova, Gardiner, Goldberg, Drummond. A protocol for isolation and visualization of yeast nuclei by scanning electron microscopy (SEM). Nature Protocols. August 2007. 2. 1943-1953.
12. Yin Y, Jiang Y, Lam K G, Berletch J B, Disteche C M, Noble W S, Steemers F J, Camerini-Otero R D, Adey A C, Shendure J. High-Throughput Single-Cell Sequencing with Linear Amplification. Mol Cell. 2019 Nov. 21; 76 (4): 676-690.e10. doi: 10.1016/j.molcel.2019.08.002. Epub 2019 Sep. 5. PMID: 31495564; PMCID: PMC6874760.

1. A method comprising:
a) dividing a plurality of fixed and permeabilized cells into a plurality of wells, each well comprising a first set of barcoding primers comprising:
(i) a universal linker strand (ULS) sequence; wherein the primers in each well comprise the same ULS sequence;
(ii) a first well-specific barcode (1-BC); wherein the primers in each well comprise a different 1-BC sequence; and a targeting region comprising at least one of:
(iii) random hexamers sequences; wherein the random hexamers sequences hybridize to complementary sequences on genomic DNA of the cells; and
(iv) specific sequences, wherein the specific sequences hybridize to target sequences on the genomic DNA of the cell;
b) amplifying genomic DNA while it remains inside of each cell to create barcoded molecules under conditions that maintain cellular membrane integrity;
c) pooling the cells from the plurality of wells;
d) dividing the cells into a plurality of wells, each well comprising a second set of barcoding primers comprising:
(v) an adapter sequence comprising a sequence complementary to the ULS sequence;
(vi) a second well-specific barcode (2-BC); wherein the primers in each well comprise a different 2-BC sequence; and
(vii) the ULS sequence wherein the primers in each well comprise the same ULS sequence;
e) amplifying the barcoded molecules under conditions that maintain cellular membrane integrity;
f) repeating steps d) through f) for a plurality of rounds, wherein the well-specific barcode is different in each round, and wherein in the final round, the set of barcoding primers further comprises an affinity moiety to generate barcoded amplicons comprising the affinity moiety;
g) lysing the cells to release the barcoded amplicons comprising the affinity moiety;
h) contacting the barcoded amplicons comprising the affinity moiety with a capture reagent; and
i) amplifying the barcoded amplicons off of the affinity moiety and affinity capture reagent to generate free amplification products.

2. The method of claim 1, wherein the target region comprises specific sequences, and wherein the specific sequences include forward primers and reverse primers for the target sequence; and wherein the method further comprises ligating a double-stranded DNA sequence comprising a terminal primer sequence to a free end of the barcoded amplicons before amplifying the barcoded amplicons off of the affinity moiety and affinity capture reagent.

3. The method of claim 1, wherein the barcoding primers comprise specific sequences, and wherein the method further comprises converting the barcoded amplicons into double-stranded amplicons by contacting the barcoded amplicons with a polymerase, and amplification primers that hybridize to segments of the barcoded amplicons complementary to the specific sequences; and performing an amplification reaction.

4. The method of claim 1, further comprising converting the barcoded amplicons into double-stranded amplicons by contacting the barcoded amplicons with a polymerase, and oligonucleotides, wherein the oligonucleotides comprise random hexamers and a terminal primer sequence, wherein the oligonucleotides are configured to produce double-stranded barcoded amplicons comprising the terminal primer sequence.

5. The method of claim 1, wherein the barcoding primers comprise only random hexamer sequences.

6. The method of claim 1, wherein the barcoding primers comprise only specific sequences.

7. The method of claim 1, wherein the barcoding primers comprise both random hexamer sequences and specific sequences.

8. The method of claim 1, wherein the amplification of step b) comprises an isothermal amplification reaction.

9-14. (canceled)

15. The method of claim 8, wherein step b) comprises contacting the plurality of fixed and permeabilized cells with an isothermal polymerase.

16. The method of claim 15, wherein the isothermal polymerase is phi29.

17. (canceled)

18. The method of claim 1, wherein step b) comprises contacting the plurality of fixed and permeabilized cells with a crowding agent.

19-30. (canceled)

31. The method of claim 3, wherein the step of converting the barcoded amplicons into double stranded amplicons comprises performing an isothermal amplification reaction.

32-46. (canceled)

47. The method of claim 1, wherein amplifying the barcoded amplicons in step i) comprises performing polymerase chain reaction (PCR).

48. The method of claim 47, wherein primers that hybridize to the terminal primer sequence are used in the PCR.

49. The method of claim 47, wherein primers that hybridize to the target sequences are used in the PCR.

50. The method of claim 1, further comprising 1) purifying the free amplification products.

51. (canceled)

52. The method of claim 1, further comprising sequencing the free amplification products.

53. The method of claim 1, wherein the ULS sequence is different in each round, and wherein the adapter in each round is complementary to the ULS sequence of the previous round.

54-74. (canceled)

75. A method comprising:
a) capturing barcoded amplicons comprising an affinity moiety by contacting the amplicons with an affinity capture reagent;
b) converting the barcoded amplicons into double-stranded captured amplicons;
c) amplifying the double-stranded captured amplicons to generate free amplification products that are not attached to the affinity moiety and affinity capture reagent.

76-104. (canceled)

105. A kit for amplifying genomic DNA within cells, the kit comprising:
a) a first plurality of barcoding primers comprising:
(i) a universal linker strand (ULS) sequence; wherein each of the plurality of primers comprises the same ULS sequence;
(ii) a first barcode (1-BC); wherein each of the plurality of primers comprises a different 1-BC sequence; and a targeting region comprising at least one of:
(iii) random hexamers sequences; wherein the random hexamers sequences hybridize to complementary sequences on genomic DNA; and
(iv) specific sequences, wherein the specific sequences hybridize to target sequences on genomic DNA; and
an isothermal polymerase.

106. The kit of claim 105, further comprising:
b) multiple sets of a second plurality of barcoding primers comprising:
(v) an adapter sequence comprising a sequence complementary to the ULS sequence;
(vi) a second barcode (2-BC); wherein each of the plurality of primers comprises a different 2-BC sequence; and wherein each set comprises a different plurality of barcoding primers; and
(vii) the ULS sequence each of the plurality of primers comprises the same ULS sequence;
c) a third plurality of barcoding primers comprising:
(viii) an adapter sequence comprising a sequence complementary to the ULS sequence;
(ix) a third barcode (3-BC); wherein each of the plurality of primers comprises a different 3-BC sequence;
(x) the ULS sequence each of the plurality of primers comprises the same ULS sequence; and
(xi) an affinity moiety.


SEQUENCE LISTING (8 sequences)

SEQ ID NO: 1 - DNA, 35 bp, synthetic construct
  source 1..35: mol_type = other DNA
atccacgtgcttgagactcgtaannnnnnataagc

SEQ ID NO: 2 - DNA, 21 bp, synthetic construct
  source 1..21: mol_type = other DNA
ttaatatggactaaaggaggc

SEQ ID NO: 3 - DNA, 21 bp, synthetic construct
  source 1..21: mol_type = other DNA
gtacttaggatacttatctga

SEQ ID NO: 4 - DNA, 21 bp, synthetic construct
  source 1..21: mol_type = other DNA
gtacttaggatacttnnnnnn

SEQ ID NO: 5 - DNA, 74 bp, synthetic construct
  source 1..74: mol_type = other DNA
catggtacccctgtacgagtgctaattaaagtagcaactgtacttaggatactttagactagcgatcgtgactg

SEQ ID NO: 6 - DNA, 10 bp, synthetic construct
  source 1..10: mol_type = other DNA
cacgattaat

SEQ ID NO: 7 - DNA, 10 bp, synthetic construct
  source 1..10: mol_type = other DNA
tgacatgaat

SEQ ID NO: 8 - protein, 55 aa, synthetic construct
DDRQGAIDVDVQLLENEVHHDVVKSKISVICESGDSAIIRSSGNSSPLFDTFIEI